(19) United States
(12) Patent Application Publication
(10) Pub. No.: US 2012/0259432 A1
(43) Pub. Date: Oct. 11, 2012

(54) Above-the-Knee Modular Prosthesis System

(76) Inventor: Timothy Dillingham, Merion Station, PA (US)

(21) Appl. No.: 13/274,146

(22) Filed: Oct. 14, 2011

Related U.S. Application Data
(63) Continuation-in-part of application No. 13/083,403, filed on Apr. 8, 2011.
Application No. 13/083,403 is a continuation-in-part of application No. 13/274,130, filed on Oct. 14, 2011.

(51) Int. Cl. A61F 2/60; A61F 2/78

(52) U.S. Cl. 623/31; 623/36

(57) ABSTRACT

An above-the-knee modular prosthetic system contains an outer housing and connector assembly which may both be adjusted to create a custom-like fit for a residual limb. The outer housing contains an inner liner which provides circumference and volume adjustability, while an outer shell adjusts for circumference of a residual limb only. A plurality of additional adjustment components may provide additional customization for the volume, circumference, shape, angle and other physical properties of a residual limb. A connector assembly connects the outer housing to a knee, shank and foot known in the art. The connector assembly contains an upper plate and a lower plate which are adjustably connected to provide adjustments for the angle and position at which a residual limb is connected to a prosthetic limb.

### CROSS-REFERENCE TO RELATED APPLICATION

This application is a continuation-in-part of U.S. patent application Ser. No. 13/083,403 entitled “Modular Prosthesis System,” filed on Apr. 8, 2011, and U.S. patent application Ser. No. 13/274,130 entitled “Rapid Fit Modular Prosthetic Device for Accommodating Gait Alignment and Residual Limb Shape and Volume,” filed on Oct. 14, 2011, which are hereby incorporated by reference in their entirety.

### FIELD OF INVENTION

The present invention relates to the field of prostheses, and more particularly to a modular prosthesis system for above-the-knee amputees.

### BRIEF DESCRIPTION OF THE DRAWINGS

FIG. 1 illustrates an exemplary embodiment of an above-the-knee modular prosthesis system.

FIG. 2 illustrates an alternative embodiment of an above-the-knee modular prosthesis system with soft inner liner.

FIGS. 3a and 3b illustrate exemplary connector assemblies for a modular prosthesis system.

FIG. 4 is an exploded view of an exemplary above-the-knee modular prosthesis system.

### GLOSSARY

As used herein, the term “closure component” refers to any component which adjusts for the circumference of a residual limb to secure an outer housing.

As used herein, the ter “connector tube” refers to any off-the-shelf 27-50 millimeter tube known in the art for use with a prosthetic limb, such as Safety Knee™.

As used herein, the term “dynamic stress point profile” refers to the unique anatomic and physiologic characteristics of an amputee's residual limb which govern the distribution of forces and stresses on the residual limb during activity.

As used herein, the term “gait” means an individual's walking pattern, including all forces which could impact a residual limb.

As used herein, the term “grid pattern” refers to a configuration of uniformly repeating shapes arranged in a network of uniformly spaced horizontal and perpendicular lines.

As used herein, the term “modular” refers to components that are interchangeable and designed to function together as a unit. Components of a modular prosthesis system may be off-the-shelf or custom-made.

As used herein, the term “off-the-shelf knee joint” refers to a standard connector tube type prosthetic knee joint having an approximately 30 millimeter pipe which is commercially available. An off-the-shelf knee joint may be a low-cost foot and knee joint component known in the art that only needs to be adjusted for height.

As used herein, the term “shank” refers to a tubular component attached to a knee mechanism at one end and a prosthetic foot at the other end.

As used herein, the term “supporting component” refers to a component which provides additional foundation for bearing the weight of the central plate and upper assembly of a connector as well as the weight of an amputee.

As used herein, the term “washer” refers to a component which distributes pressure from another component and provides a firm attachment through friction to prevent movement of the component. For example, a washer placed under a threaded fastener will distribute the pressure from the head of the fastener and prevent movement of the fastener.

### BACKGROUND

Over 150,000 amputations occur in the United States annually. Amputations are rising in frequency due to diabetes and peripheral vascular disease. The transfemoral (above knee) level of amputation is less common than the below knee (transtibial) level of limb loss, but results in the highest level of gait dysfunction and disability. Further, the transfemoral level is difficult to fit with a prosthetic socket due to redundant soft tissues and variable lengths and sizes of the residual limb.

A transfemoral prosthesis is an artificial limb that replaces the portion of the leg above the knee that is missing. The shape of the residual limb varies for each individual and generally requires a custom-fitted prosthesis. A comfortable custom-fitted prosthesis is difficult to fabricate and costly to provide using conventional manufacturing techniques.

The initial cost of a conventional prosthesis for a transfemoral amputee typically ranges from $10,000 to $20,000 depending upon the components used and the difficulty in fitting the individual. In addition, there are additional costs to ensure the comfort and functionality of the device including replacement or revision of the socket. The present state of prosthesis fabrication often requires three or more visits to the prosthetist and there are multiple steps in the fabrication process. First, a cast mold of the residual limb is made and a positive cast that resembles the residual limb is generated. Then, a prosthetic socket is built to custom-fit over the positive cast. Sometimes a check or temporary socket is made to insure a better fit. Typical fabrication techniques require specialized facilities. Generally, the final prosthesis requires post-fabrication adjustments as the residual limb tissue changes over time.

Recent advancements have been made in the field of prosthetic devices. However, devices such as computerized knee mechanisms and energy storing feet are costly and beyond the economic means of many prosthetic users, particularly those in nations outside the United States. Insurance coverage of such prosthetic devices is variable across insurers and has often impeded prescription and availability of high quality devices even for amputees with insurance coverage. The uninsured often go without comfortable prosthetic devices for long periods of time before public insurance enables them to receive a functional prosthesis.

Attempts have been made in the prior art to develop prosthesis systems that can be globally manufactured and distributed. These prosthesis systems, however, have several limitations. They are difficult to fabricate and require specialized facilities for initial manufacturing (e.g., casting) and subsequent adjustments. These systems all require expertise and consulting support that is not widely available. In particular, the socket (i.e., the portion of the prosthesis into which the residual limb fits), socket attachment, and alignment aspects of the device seem to be a common problematic area of development.

It is desirable to create a prosthetic device which eliminates the need for complex fabrication and specialized tools or labs, and which can be economically manufactured and distributed on a global basis.

It is desirable to create a prosthetic device which is immediately fit and aligned on the residual limb during the initial clinical visit and is adjustable and modular to accommodate different residual limb sizes and volume fluctuations that frequently occur in patients after amputation or those with heart failure and renal diseases.

It is desirable to create a prosthetic device which is one size and adjustable to fit many shapes.

### SUMMARY OF THE INVENTION

The present invention is a modular prosthesis system comprised of an outer housing, connector assembly and prosthetic shank. The connector is made up of two main components: an upper plate which is secured to the outer housing and a lower plate which is secured around a pipe connector that attaches to the knee device. The design of the connector allows for angular adjustment which ensures proper positioning and alignment of the knee, shank, and foot. In addition, the housing includes a liner and tightening components, resulting in a prosthesis that may be fit to any residual limb and which can accommodate long-term and daily changes in the amputee and residual limb as well as other aspects of an amputee's dynamic stress distribution profile.

### DETAILED DESCRIPTION OF INVENTION

For the purpose of promoting an understanding of the present invention, references are made in the text to exemplary embodiments of a modular prosthesis system, only some of which are described herein. It should be understood that no limitations on the scope of the invention are intended by describing these exemplary embodiments. One of ordinary skill in the art will readily appreciate that alternate but functionally equivalent materials, component, and designs may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or“approximately” as used herein may be applied to modify any quantitative representation that could permissibly vary without resulting in a change in the basic function to which it is related.

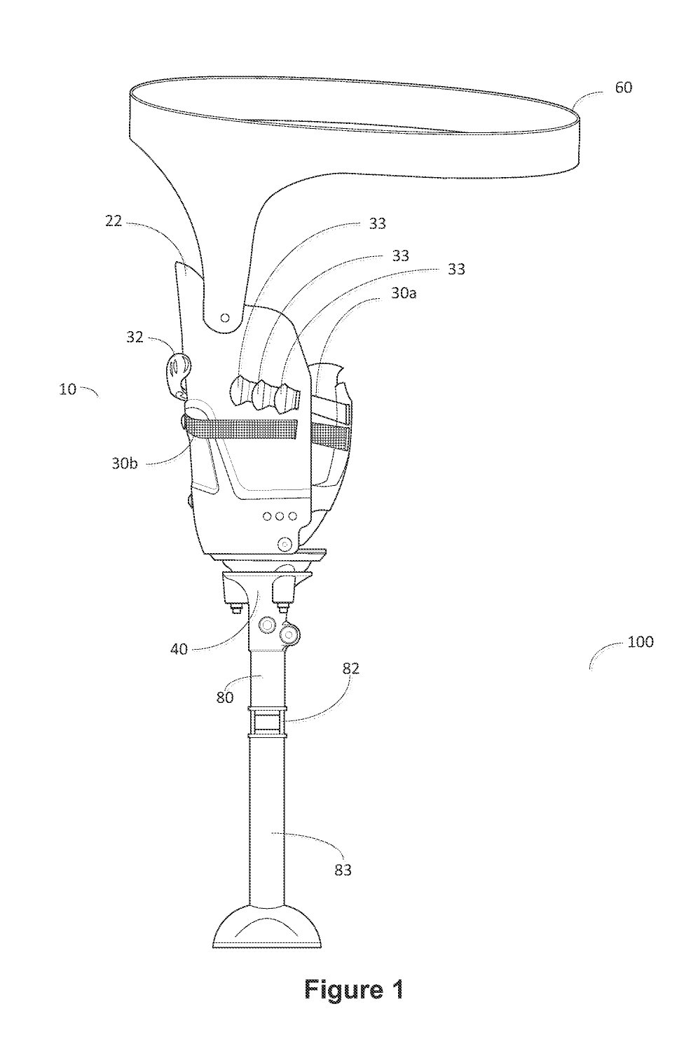

FIG. 1 illustrates an exemplary embodiment of an above-The-knee modular prosthesis system 100. As illustrated in FIG. 1, modular prosthesis system 100 includes universal outer housing 10, consisting of soft inner liner 20 (not shown) and outer shell 22.

Closure components 30a, 30b on outer shell 22 allow outer shell 22 to be adjusted to the circumference of an amputee's residual limb. In the exemplary embodiment shown, closure component 30a is a looped wire running from buckle 32 to secure around one of hook-shaped protuberances 33, and closure component 30b is a strong non-elastic strap completely encircling outer shell 22 and serves as a safety strap. In further exemplary embodiments, outer shell 22 may contain any number of closure components, and closure components may be any structure or device known in the art to allow width adjustability of outer shell 22. For example, closure components may include, but are not limited to, buttons, snaps, clasps, clips, elastic components, buckles, laces, ties, interlocking components, hook-and-loop fasteners, hook-and-eye fasteners, hook-shaped components, and any combination these and other structures and devices.

Universal outer housing 10 releasably secures to connector assembly 40 and connecting tube 80. Suspension component 60, which in the exemplary embodiment shown is a waist strap, helps an amputee more securely hold modular prosthesis system 100 to a residual limb. In further exemplary embodiments, suspension component 60 may be any adjustable securing component or device known in the art, including, but not limited to, suspenders, belts, clasps or other attachment means which releasably attach to a user's clothing or existing belt, or any combination of these and other structures.

In some exemplary embodiments, suspension component 60 may contain additional elements to create a suspension system. For example, a liner or sleeve which fits over a residual limb may be provided with suspension component 60. In further exemplary embodiments, a liner or sleeve may include a cushioning gel substance or other component. In still further embodiments, a liner or sleeve may contain directionally frictional materials which allow the liner or sleeve to easily slide into outer housing 10, but require additional force to be removed from outer housing 10.

In yet further exemplary embodiments, outer housing 10 and connector assembly 40 may be adapted to accommodate a liner or sleeve with a serrated pin suspension system, such as the ALPS pin and gel liner suspension system known in the art.

In the exemplary embodiment shown, universal outer housing 10 is a single unit constructed of rigid plastic. In further exemplary embodiments, outer housing 10 may be multiple separate components molded or joined together, such as with closure components 30. In still other exemplary embodiments, outer housing 10 may be constructed of a stronger material, such as metals, or materials specifically designed to withstand the pressure and wear caused by an amputee's activities. Closure components 30 may be selected based on the material of outer housing 10 or the specific forces generated by an individual amputee's residual limb.

In the exemplary embodiment shown in FIG. 1, connector tube 80 connects to prosthetic knee 82, which connects to below-the-knee shank 83, which is a standard below-the-knee shank known in the art and provides height adjustment for the distance from knee 82 to the prosthetic foot which contacts the ground.

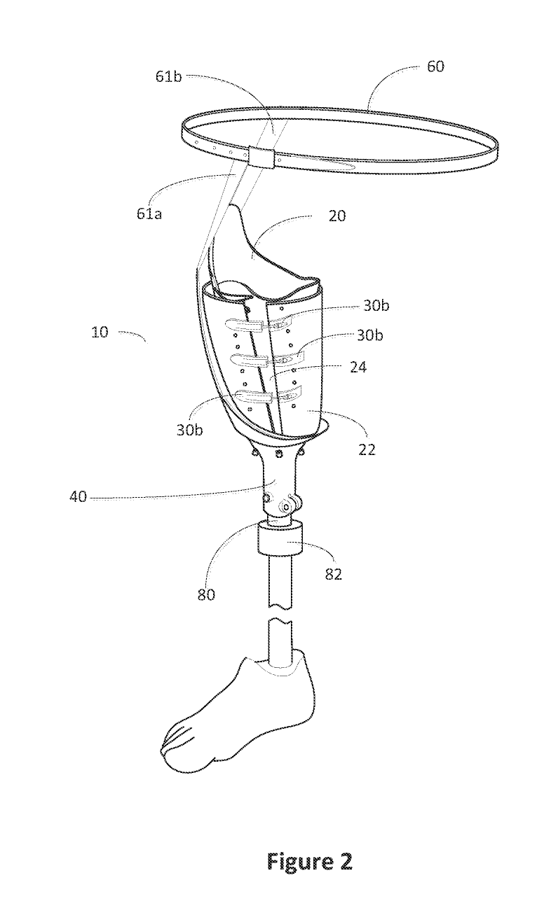

FIG. 2 illustrates an alternative embodiment of an above-the-knee modular prosthesis system 100 with soft inner liner 20. In the exemplary embodiment shown, soft inner liner 20 fits within the cavity created by outer shell 22 and provides comfortable support for a residual limb. Soft inner liner 20 may also decrease the internal volume of the cavity created by outer shell 22 to help accommodate a residual limb having a smaller circumference.

In the exemplary embodiment shown, inner liner 20 is created of a deformable material, such as cushion, foam, gel or other pillow-like material which deforms to specifically contour a residual limb. In other exemplary embodiments, inner liner 20 may be custom-made to fit a specific residual limb.

As illustrated in FIG. 2, suspension component 60 is a belt with two side straps 61a, 61b which attach to outer housing 10. In other exemplary embodiments, side straps 61a, 61b may be attached to inner liner 20.

In the exemplary embodiment shown, outer housing 10 contains three identical closure components 30b which are buckles. Closure components 30b tighten against outer shell 22 to close gap 24 and apply pressure around a residual limb to keep it in outer housing 10. In further exemplary embodiments, closure components may each be different. In yet further exemplary embodiments, closure components may be specifically designed or positioned to apply pressure at specific points around a residual limb.

Outer housing 10 attaches to connector 40, which in the exemplary embodiment shown is adjustable for making angular adjustments. For example, connector 40 may be able to tilt backwards, forwards and/or to the sides to account for differences in an individual's gait and natural bone alignment. Connector 40 provides adjustment of the angle of the prosthesis and leg on the amputee to optimally align the prosthesis. After it is adjusted and put into the proper position angle, connector 40 is tightly secured in place such that it provides a stable and non-movable attachment for safe ambulation.

Connector 40 attaches outer housing 10 to connector pipe 80, which is a standard diameter pipe connector known in the art. In the exemplary embodiments shown in FIGS. 1 and 2, connector 80 is drawn attached to knee component 82, which connects via shank 83 to a foot-like prosthetic limb. In further exemplary embodiments, shank 83 may be any prosthetic shank known in the art.

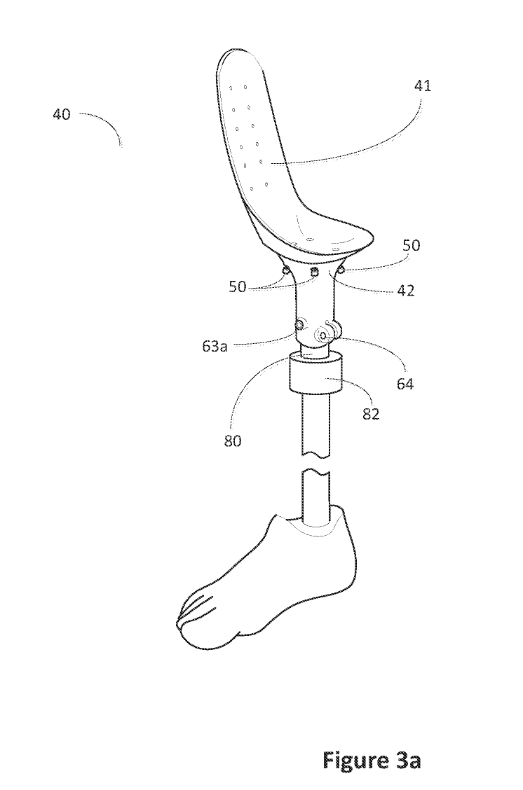

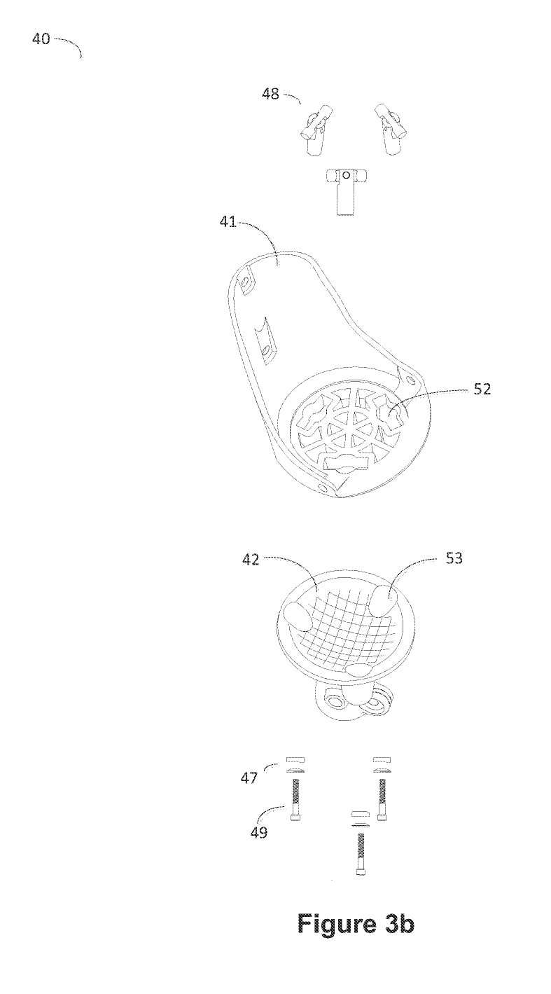

FIGS. 3a and 3b illustrate exemplary connector assemblies 40 for a modular prosthesis system 100.

As illustrated in FIG. 3a, connector assembly 40 includes upper plate 41 and lower plate 42 joined by bolts 50. Lower plate 42 contains an inner tubular recess for receiving connector pipe 80. Set screws 63a and 63b and tightening bolt 64 help tighten connector pipe 80 to connector assembly 40.

Bolts 50 allow for gait adjustability. When a residual limb is secured in outer housing 10 (not shown), outer housing 10 (not shown) is securely attached to upper plate 41. Upper plate 41 and lower plate 42 may be pivotally adjustable, relative to each other, to conform modular prosthesis system 100 to a specific individual. In further exemplary embodiments, upper plate 41 and lower plate 42 may contain a limited degree of rotational adjustability. Bolts 50 allow an amputee to account for differences in bone structure, curvature, and alignment.

FIG. 3b is an alternative exemplary connector assembly 40. Upper plate 41 connects to lower plate 42 using specialized bolts comprised of a hollow, pivotal female end 48 with threaded male end 49. Washers 47 may be optionally included with male end 49. Pivotal female ends 48 project downwards through bolt apertures 52, and male ends 49 project upwards through corresponding bolt channels 53 to engage female ends 48. Pivotal female ends 48 allow limited movement and adjustability of upper plate 41 relative to lower plate 42.

In the exemplary embodiment illustrated, once a desired position has been reached, the pivotal bolts assemblies may be tightened into place, permanently or adjustably, to prevent upper plate 41 and lower plate 42 from moving under the forces exerted by a residual limb and movement of an amputee. In further exemplary embodiments, upper plate 41 and lower plate 42 may be secured together with a limited amount of allowable movement for such things as absorbing excessive gait forces.

While upper plate 41 and lower plate 42 are illustrated as joined by three pivotal bolt assemblies, in further exemplary embodiments, upper plate 41 and lower plate 42 may be adjustably attached through any structure or device known in the art, including, but not limited to, screws, pins, bolts, interlocking components, or any combination of these and other structures or devices.

Both FIGS. 3a and 3b show different structures to provide limited adjustability of modular prosthetic system 100 to account for differences in bone structure, shape and alignment, as well as differences in gait, to create a custom-like fit for each amputee.

In some exemplary embodiments, as illustrated in FIG. 3b, upper plate 41 and/or lower plate 42 may contain surface textures which may facilitate or incrementally limit the adjustability of connector assembly 40. As shown in FIG. 3b, lower plate 42 contains a grid pattern which corresponds to a similar grid pattern on the under-surface of upper plate 41. The corresponding grid patterns create a plurality of locations to which connector assembly 40 may be positioned. When the grid-like surfaces connect, the position is more stable and resistant to change when experiencing the various forces applied to connector assembly 40 by a residual limb and the general movement of an amputee.

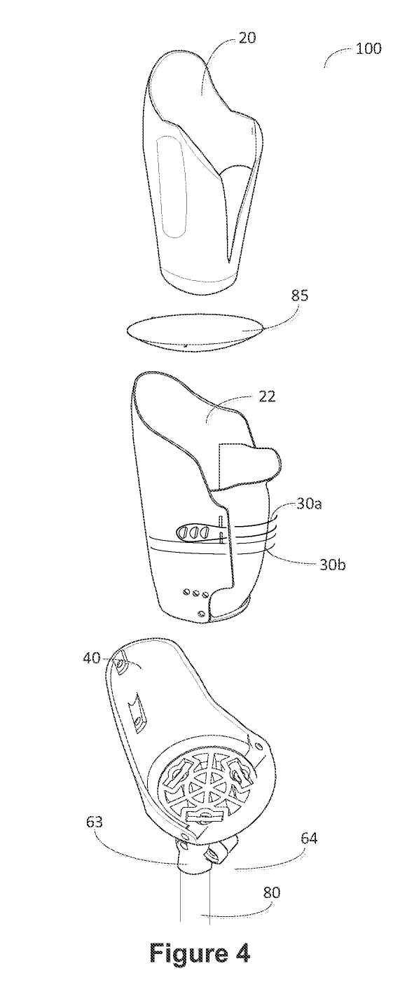

FIG. 4 is an exploded view of an exemplary above-the-knee modular prosthesis system 100. Soft inner liner 20 is removed from outer shell 22. In some exemplary embodiments, soft inner liner 20 may contain closure components, such as laces, buckles, hook-and-eye fasteners, hook-and-loop fasteners or other structures or combination of structures known in the art to secure soft inner liner 20 around a residual limb. As illustrated, outer shell 22 contains closure components 30a, 30b, which are a looped cable and securing band, as described in FIG. 1. Height adjustment component 85 is shown between inner liner 20 and outer shell 22. Connector assembly 40 contains connector 80, rotationally and vertically secured in place by set screw 63a and tightening bolt 64.

In the exemplary embodiment shown, height adjustment component 85 is a plate which may be positioned within outer shell 22 to adjust for the distance between a residual limb and the natural location of a knee joint. As illustrated, height adjustment component 85 is friction-fit within outer shell 22. In further exemplary embodiments, height adjustment component 85 may contain pins, bolts, or other structures adapted to project through outer shell 22, creating a more permanent adjustment. In still further exemplary embodiments, outer shell 22 may contain a plurality of pre-determined height-adjustment locations to which height adjustment component 85 may be secured.

In further exemplary embodiments, height adjustment component 85 may also be used to adjust to the angle of a residual limb and therefore alter the angle at which modular prosthetic system 100 is attached. For example, height adjustment component 85 may be pivotally attached to outer shell 22, or secured to outer shell 22 at an angle.

In some exemplary embodiments, height adjustment component 85 may be made of a solid material, such as plastics or metals. In further exemplary embodiments, height adjustment component 85 may contain a form of cushioning or padding to decrease the pressure on a residual limb. However, height adjustment component 85 will need to be able to support the weight of an amputee.

In further exemplary embodiments, when height adjustment is not necessary, height adjustment component 85 may be omitted. In yet further exemplary embodiments, an additional cushion or padded component may be placed between inner liner 20 and outer shell 22.

In yet further exemplary embodiments, inserts and adjustment components of various shapes, sizes and contours may be added to adjust for a residual limb's circumference, volume, size, angle, and other properties. For example, modular prosthetic system 100 may include height adjustment components, volume adjustment components, angle adjustment components, circumference adjustment components and combinations of such adjustment components. By providing modular adjustment components, modular prosthetic system 100 may be manufactured in a standard size, or select standard sizes, yet adjusted to provide a near custom fit for each residual limb. For example, universal outer housing 10 may be manufactured in three sizes, with variations in soft inner liner 20 and height adjustment component 85 and the adjustability provided by closure components 30 and other components creating a wide range of sizes.

In the exemplary embodiments described, components of modular prosthetic system 100 may be disposable. For example, the various liners, pads and adjustment components may be specifically designed to be quickly and easily changed and disposable as an amputee's residual limb changes size or shape. In other exemplary embodiments, components of modular prosthetic system 100 which experience wear may be designed to be replaced and disposed as they weaken.

In other exemplary embodiments, components of modular prosthetic system 100 may be specifically designed and manufactured for efficient shipping. For example, liners, shells and other components may be specifically designed to nest within each other, saving room during shipping. Other components, such as bolts, screws and closure components, may also be assembled for shipping.

Modular prosthetic system 100 also allows a prosthetic limb to be quickly and securely attached to a residual limb. The adjustability of the various components provides a quick way to create a custom-like fit by accounting for differences in residual limb shape, circumference, volume and general size, as well as differences in gait, bone structure and bone alignment. Because it is not necessary to create custom pieces or molds, modular prosthetic system 100 may be implemented immediately.

What is claimed is:

1. A modular prosthetic system for transfemoral residual limbs comprising:
an outer housing comprising
an outer shell,
at least one closure component,
at least one waist strap component, and
at least one adjustment component; and
a connector comprising
an upper plate,
a lower plate, and
a hollow tubular component with an aperture adapted to receive a connector pipe,
wherein said upper plate and said lower plate are adjustably connected.

2. The modular prosthetic system of claim 1 wherein said at least one adjustment component is selected from the group consisting of an inner liner, height adjustment component, a volume adjustment component, an angle adjustment component, a circumference adjustment component and combinations thereof.

3. The modular prosthetic system of claim 1 which further includes a waist strap component.

4. The modular prosthetic system of claim 3 wherein said waist strap includes at least one suspension component.

5. The modular prosthetic system of claim 1 wherein said closure component provides adjustable tension of said outer housing around a residual limb.

6. The modular prosthetic system of claim 1 which further includes an inner liner which conforms to the shape of a residual limb.

7. The modular prosthetic system of claim 1 wherein said upper plate and said lower plate are connected by a plurality of pivotable bolts.

8. The modular prosthetic system of claim 1 wherein the lower surface of said upper plate and the upper surface of said lower plate contain corresponding grid patterns.

9. The modular prosthetic system of claim 1 wherein said upper plate and said lower plate are adjustably connected to provide forward adjustment, backward adjustment and side-to-side adjustment.

10. The modular prosthetic system of claim 1 wherein said connector pipe is a standard connector pipe known in the art having a diameter of approximately 27 to 50 millimeters.

11. The modular prosthetic system of claim 1 which contains a plurality of closure components.

12. The modular prosthetic system of claim 1 wherein said at least one closure component is selected from the group consisting of buttons, snaps, clasps, dips, elastic components, buckles, laces, ties, interlocking components, hook-and-loop fasteners, hook-and-eye fasteners, hook-shaped components, looped wires and combinations thereof.

13. A modular prosthetic system for transfemoral limbs comprising:
an outer housing comprising
an outer shell,
an inner liner,
at least one circumference adjusting closure component,
at least one waist strap component, and
at least one additional adjustment component;
a connector comprising
an upper plate,
a lower plate,
a hollow tubular component with an aperture, and
an above-the-knee connector pipe connected at one end to said hollow tubular component by insertion into said aperture and connected at the other end to a knee joint,
wherein said upper plate and said lower plate are adjustably connected to provide frontwards, backwards and side-to-side adjustment; and
a below-the-knee shank which provides height adjustment connected to said knee joint and a prosthetic foot.

14. The modular prosthetic system of claim 13 wherein said at least one additional adjustment component is a height adjustment component.

15. The modular prosthetic system of claim 13 wherein said at least one additional adjustment component is selected from the group consisting of a height adjustment component, a volume adjustment component, an angle adjustment component, a circumference adjustment component and combinations thereof.

16. The modular prosthetic system of claim 13 wherein said above-the-knee connector pipe is a standard connector pipe known in the art having a diameter of approximately 27 to 50 millimeters.

17. The modular prosthetic system of claim 13 wherein said below-the-knee shank is a standard shank known in the art having a diameter of approximately 27 to 50 millimeters.

18. The modular prosthetic system of claim 13 wherein said inner liner provides circumference and shape adjustment.

19. The modular prosthetic system of claim 13 wherein the lower surface of said upper plate and the upper surface of said lower plate have corresponding textures for forwards, backwards and side-to-side adjustment.

20. The modular prosthetic system of claim 13 wherein said outer housing is manufactured in at least one predetermined size and said at least one additional adjustment component provides personalized adjustment.
